(19) United States
(12) Patent Application Publication
(10) Pub. No.: US 2010/0016700 A1
(43) Pub. Date: Jan. 21, 2010

(54) ANALYTE MEASUREMENT AND MANAGEMENT DEVICE AND ASSOCIATED METHODS

(75) Inventors: Zara Sieh, Pleasanton, CA (US); David Horwitz, Los Altos, CA (US); David Price, Pleasanton, CA (US); Peter Krulevitch, Pleasanton, CA (US); Donna Savage, Rolling Hills Estates, CA (US); Robert Shartle, Arnold, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan, Inc., Milpitas, CA (US)

(21) Appl. No.: 12/505,007

(22) Filed: Jul. 17, 2009

Related U.S. Application Data
(60) Provisional application No. 61/082,106, filed on Jul. 18, 2008.

(51) Int. Cl. A61B 5/00; G06F 3/048; H04L 9/32; G08B 1/08; G06F 15/16; G06N 5/02

(52) U.S. Cl. 600/365; 715/810; 726/16; 340/539.12; 709/202; 709/206; 706/54

(57) ABSTRACT

Various embodiments of a diabetes management system are provided. One exemplary system may include an analyte measurement device and a therapeutic agent delivery device. The measurement device includes a measurement unit, display, and first wireless module. The therapeutic agent delivery device has a delivery device housing, delivery mechanism disposed in the housing that delivers a dosage of the agent to the user upon actuation by the user or health care provider, and a second wireless module. The second module, automatically, without prompting from a user or any active input or action by the user, transmits a signal to the first wireless module indicative of: (a) type of therapeutic agent delivered; and (b) amount of therapeutic agent delivered to the user; or (c) type of therapeutic agent device from which the therapeutic agent was administered. Also described are diabetes management devices and methods.

### PRIORITY

This application claims the benefits of priority under 35 USC § 119 to U.S. Provisional Patent Application Ser. No., 61/082,106 filed on Jul. 18, 2008, which application is incorporated by reference in their entireties herein this application.

### BACKGROUND

Introduction and management of insulin therapy to a patient with Type 2 diabetes can be overwhelming to the patient and a burden to the provider due to the complexity of conventional methods and devices for doing so. Significant training of the patient may be necessary. The patient may need to learn, for example, various concepts and actions including hypoglycemia management, injections and the proper use insulin administration devices, as well as the mechanical, electronic, and software aspects of using a blood glucose meter. In addition, the patient must learn to follow the doctor's instructions in starting and adjusting insulin dosages on a regular basis (e.g. per meal, daily, 2× weekly, or weekly basis).

Detailed instructions as to the prescribed blood glucose testing and insulin titration protocol are typically written out by the health care professional or checked off on a piece of paper. Patients often keep handwritten logs in order to comply.

After getting onto insulin therapy, a patient often times presents in a physician's office with poor glycemic control and the care provider (i.e., physician) can be left guessing as to whether the poor glycemic control is due to, for example, noncompliance, or whether increased intensification of insulin therapy is required, or a combination thereof.

### SUMMARY OF THE DISCLOSURE

Applicants have recognized the shortcomings and have therefore provided for an invention to resolve these shortcomings. In one embodiment, a diabetes management system is provided that includes an analyte measurement device and a therapeutic agent delivery device. The measurement device has a housing, processor and memory disposed in the housing. The measurement device includes a measurement unit, display, and first wireless module. The measurement unit is in communication with the processor to provide a numerical value representing generally an amount of analyte in body fluids. The display is in communication with the processor to display information relating to analyte and the therapeutic agent. The first wireless module is coupled to the processor and memory to store data received by the first wireless module in the memory. The therapeutic agent delivery device has a delivery device housing, delivery mechanism disposed in the housing that delivers a dosage of the agent to the user upon actuation by the user or health care provider, and a second wireless module. The second wireless module is disposed in the housing and configured so that the second wireless module automatically, without prompting from an user or any active input or action by the user, transmits a signal to the first wireless module indicative of: (a) type of therapeutic agent delivered; and (b) amount of therapeutic agent delivered to the user; or (c) type of therapeutic agent device from which the therapeutic agent was administered.

In yet another embodiment, a diabetes management system is provided that includes an analyte measurement device, a therapeutic agent delivery device, and a healthcare provider's computer. The measurement device has a housing, processor and memory disposed in the housing. The measurement device includes a measurement unit, display, and first wireless module. The measurement unit is in communication with the processor to provide a numerical value representing generally an amount of analyte in body fluids. The display is in communication with the processor to display information relating to analyte and the therapeutic agent. The first wireless module is coupled to the processor and memory to store data received by the first wireless module in the memory. The therapeutic agent delivery device has a delivery device housing, delivery mechanism disposed in the housing that delivers a dosage of the agent to the user upon actuation by the user or health care provider, and a second wireless module. The second wireless module is disposed in the housing and coupled to the delivery mechanism, the second wireless module, upon prompting or by an action from a user, transmits a signal to the first wireless module indicative of: (a) type of therapeutic agent delivered; and (b) amount of therapeutic agent delivered to the user; or (c) type of therapeutic agent device from which the therapeutic agent was administered. The health care provider computer is in communication with the device to prescribe one of a plurality of therapeutic agent titration protocols to the memory of the device.

In a further embodiment, a diabetes management device is provided. The diabetes management device includes a housing, processor, memory, measurement unit and a display. The processor and memory are disposed in the housing, the memory includes a plurality of therapeutic administration protocols loaded into the memory from an external source that relates a dosage administration to one or more analyte amount. The measurement unit is in communication with the processor to provide a numerical value representing generally an amount of analyte in body fluids. The display is in communication with the processor to display information relating to measured amount of analyte and the therapeutic agent.

In another embodiment, a method of managing diabetes is provided. The method can be achieved by selecting a therapeutic administration protocol in accordance with therapeutic requirements of the diabetic user transferring the therapeutic administration protocol to an analyte measurement device assigned to the user; confirming delivery of therapeutic agent to the user in accordance with the therapeutic administration protocol; and generating a plurality of prompts to the user including:

(a) a reminder to measure analyte at a specified time; (b) a reminder to administer a recommended dosage of therapeutic agent within a specified time frame; and (c) a report of compliance of the user to the therapeutic administration protocol. In this method, the selecting may include entering a set up mode; selecting one of a plurality of therapeutic administration protocols, and upon selection of: (a) a long acting protocol, selecting a body weight range and confirming a starting dosage, maximum dosage, fasting measurement and specified time for delivery of the therapeutic agent; (b) a mixture protocol, selecting a frequency of delivery of therapeutic agent and confirming the frequency and specified time for delivery of the therapeutic agent; or (c) a multiple daily administration protocol, selecting a largest meal during a specified time duration and confirming the dosage of a long acting therapeutic agent at a specified time and a rapid acting therapeutic agent at a different specified time. Further, in this method, the generating includes displaying at least one of: (a) a result of the analyte measurement; (b) the dosage specified; or (c) a summary of the dosage administered at one or more specified time slots.

In yet another embodiment, a method of operating an analyte measurement device is provided. The device has a plurality of therapeutic administration protocols stored in a memory of the device. The memory is in communication with a processor, the processor configured to interface with user inputs and provide various output information. The method can be achieved by: accessing a selection menu generated by the processor with protocols loaded into the memory from an external source; selecting one therapeutic administration protocol from the plurality of therapeutic administration protocols; and outputting dosage information for therapeutic agent to be administered to a user based on one or multiple analyte amounts or concentration values stored in the memory.

These and other embodiments, features and advantages will become apparent to those skilled in the art when taken with reference to the following more detailed description of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), of which:

FIG. 1 is a simplified plan view of an analyte measurement and management device according to an embodiment of the present invention;

FIG. 2 is a simplified block diagram illustrating the internal components of an analyte measurement and management device according to an embodiment of the present invention;

FIG. 3 is a flow chart illustrating a method of operating an analyte measurement device, during which a recommended therapeutic agent dosage and a recommended time for administration of the recommended therapeutic agent dosage are calculated, according to an embodiment of the present invention;

FIG. 4 illustrates an exemplary flow chart illustrating a method of operating an analyte measurement device, in which a therapeutic agent type is entered and a list of administration protocols are displayed, according to an embodiment of the present invention;

FIG. 5 illustrates an exemplary flow chart illustrating a method of operating an analyte measurement device, in which a user's health profile is entered and a recommended therapeutic agent and administration protocol are displayed, according to an embodiment of the present invention;

FIG. 6 illustrates an exemplary flow chart illustrating a method of operating an analyte measurement device, in which an intensification administration protocol is selected, according to an embodiment of the present invention;

FIG. 7 illustrates an exemplary flow chart illustrating a method of operating an analyte measurement device, in which a recommended administration protocol is reinitialized, according to an embodiment of the present invention;

FIG. 8 is a flow chart illustrating a method of operating an analyte measurement device, in which a user is reminded to test and administer therapeutic agent if confirmation of testing or administration is not received within a time window, according to an embodiment of the present invention;

FIG. 9 is a flow chart illustrating a method of operating an analyte measurement device, in which more than one therapeutic agent is selected and more than one recommended administration protocol is displayed, according to an embodiment of the present invention;

FIG. 10 illustrates an exemplary flow chart illustrating a method of operating an analyte measurement device, in which reminders are displayed along with a compliance report, according to an embodiment of the present invention;

FIG. 11 illustrates a series of user interface screen images (displays) as can be used in methods according to various embodiments of the present invention;

FIG. 12 illustrates a user interface screen images, that assists a healthcare provider in selecting an administration protocol, that can be employed in methods according to an embodiment;

FIG. 13 illustrates a user interface image, in which a summary report is displayed, that can be employed in methods according to the present invention;

FIG. 14 illustrates an exemplary treat-to-target therapeutic administration protocol that can be used in embodiments of the present invention;

FIG. 15 illustrates another exemplary treat-to-target therapeutic administration protocol that can be employed in embodiments of the present invention;

FIG. 16 illustrates yet another exemplary treat-to-target protocol that can be utilized embodiments of the present invention;

FIG. 17 is a simplified block diagram of an analyte measurement and management device for use with a user-activated therapeutic agent delivery device according to an embodiment of the present invention; and

FIG. 18 is a flow diagram illustrating stages in a method according to an embodiment of the present invention.

FIG. 19 illustrates the various screens during a set up of a therapeutic protocol by a Health Care Provider.

FIG. 20 illustrates the various screens generated at a user's device upon selection of a first therapeutic protocol.

FIG. 21 illustrates the various screens generated at a user's device upon selection of a second therapeutic protocol.

FIGS. 22-23 illustrate the various screens generated at a user's device upon selection of a third therapeutic protocol.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. As used herein, the conjunctive “or” is not intended to have the same meaning as the logical operator or exclusive operator but is intended to include the conjunctive “and.” Furthermore, the terms “about” or “approximately” for any numerical values or ranges indicate a suitable dimensional tolerance that allows the part or collection of components to function for its intended purpose as described herein. In addition, as used herein, the terms “user”, “patient”, “host” and “subject” refer to any human or animal subject and are not intended to limit the systems or methods to human use, although use of the subject invention in a human patient represents a preferred embodiment.

Embodiments described and illustrated herein provide an analyte (e.g., blood glucose) measurement and management devices, systems, and associated methods that simplify training and guide a patient regarding when to measure an analyte (i.e., to “test”) and how much and when to administer a therapeutic agent (such as insulin) in a simple and convenient manner and with a minimum of devices. Embodiments of the analyte measurement and management device and system are also beneficial to care providers (for example, physicians) by gathering, organizing and storing information that provides insight into how effective a patient is in following a prescribed analyte management regimen.

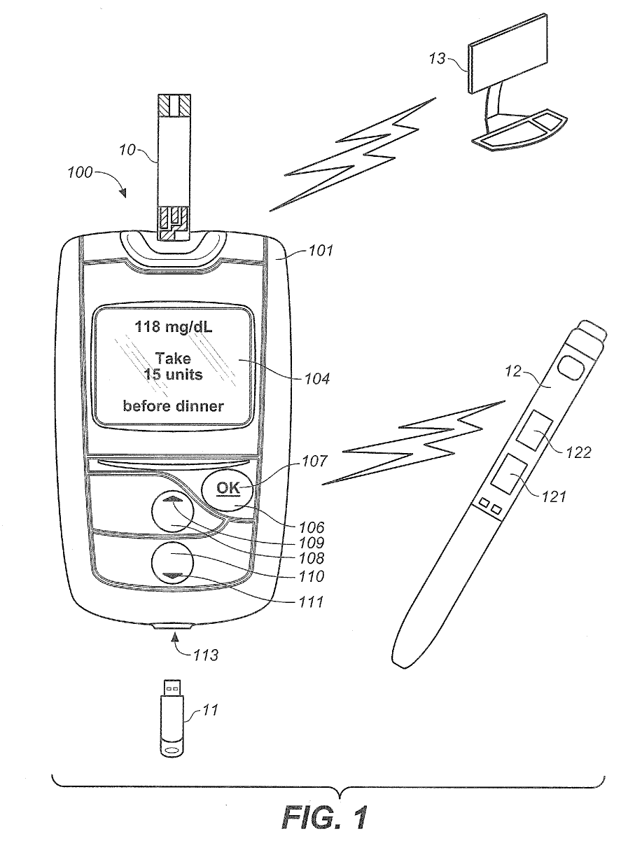

FIG. 1 illustrates an analyte measurement and management device 100 (also referred to herein for simplicity as a “meter,” an “analyte measurement device,” and a “testing device”), for testing (measuring or determining) and managing glucose levels in the blood of an individual. As is described further herein, analyte measurement and management device 100 is for use with a user-activated therapeutic agent delivery device. In this regard, the term “user-activated” refers to therapeutic delivery devices that require manual interaction between the device and a user (for example, by a user pushing a button on the device) to initiate a single therapeutic agent delivery event and that in the absence of such manual interaction deliver no therapeutic agent to the user. A non-limiting example of such a user-activated therapeutic agent delivery device is described in co-pending U.S. Provisional Application No. 61/040,024 (tentatively identified by Attorney Docket No. LFS-5180; and U.S. Provisional Application No. 61/156,386, entitled “Medical Module for Drug Delivery Pen” filed on Feb. 27, 2009 (Attorney Docket No. LFS-5196USPSP), all of which are hereby incorporated in whole by reference. Another non-limiting example of such a user-activated therapeutic agent delivery device is an insulin pen 12. Insulin pens are loaded with a vial or cartridge of insulin, and are attached to a disposable needle. Portions of the insulin pen can be reusable, or the insulin pen can be completely disposable. Insulin pens are commercially available from companies such as Novo Nordisk, Aventis, and Eli Lilly, and can be used with a variety of insulin, such as Novolog, Humalog, Levemir, and Lantus. Another insulin pen, which can be utilized herein, includes the device described and illustrated in EP0749332 entitled “Medication Delivery Device with a Microprocessor and Characteristic Monitor” which is hereby incorporated by reference in its entirety.

Analyte measurement device 100 may include user interface buttons (106, 108, 110) for entry of data, navigation of menus, and execution of commands. Data can include values representative of analyte concentration, and/or information, which are related to the everyday lifestyle of an individual. Information, which is related to the everyday lifestyle, can include food intake, medication use, the occurrence of health check-ups and general health condition and exercise levels of an individual. Analyte measurement device 100 also may include display 104. Display 104 can be used to report measured glucose levels, and to facilitate entry of lifestyle related information.

Analyte measurement device 100 may include first user interface button 106, second user interface button 108, and third user interface button 110. User interface buttons 106, 108, and 110 facilitate entry and analysis of data stored in the testing device, enabling a user to navigate through the user interface displayed on display 104. User interface buttons 106, 108, and 110 include first marking 107, second marking 109, and third marking 111, which help in correlating user interface buttons to characters on display 104.

Analyte measurement device 100 can be turned on by inserting a test strip 10 into data port 112, by pressing and briefly holding first user interface button 106, or when data traffic is detected across data port 113. Analyte measurement device 100 can be switched off by removing the test strip 10, pressing and briefly holding first user interface button 106, navigating to and selecting a meter off option from a main menu screen, or by not pressing any buttons for a predetermined time. Display 104 can optionally include a backlight.

Data port 113 accepts a suitable connector attached to a connecting lead, thereby allowing analyte measurement device 100 to be linked to an external device such as a personal computer. Data port 113 can be any port that allows for transmission of data (serial or parallel) such as, for example, serial or parallel port in wired or wireless form. A personal computer, running appropriate software, allows entry and modification of set-up information (e.g. the current time, date, and language), and can perform analysis of data collected by analyte measurement device 100. In addition, the personal computer may be able to perform advanced analysis functions, and/or transmit data to other computers (i.e. over the internet) for improved diagnosis and treatment. Connecting analyte measurement device 100 with a local or remote computer facilitates improved treatment by health care providers.

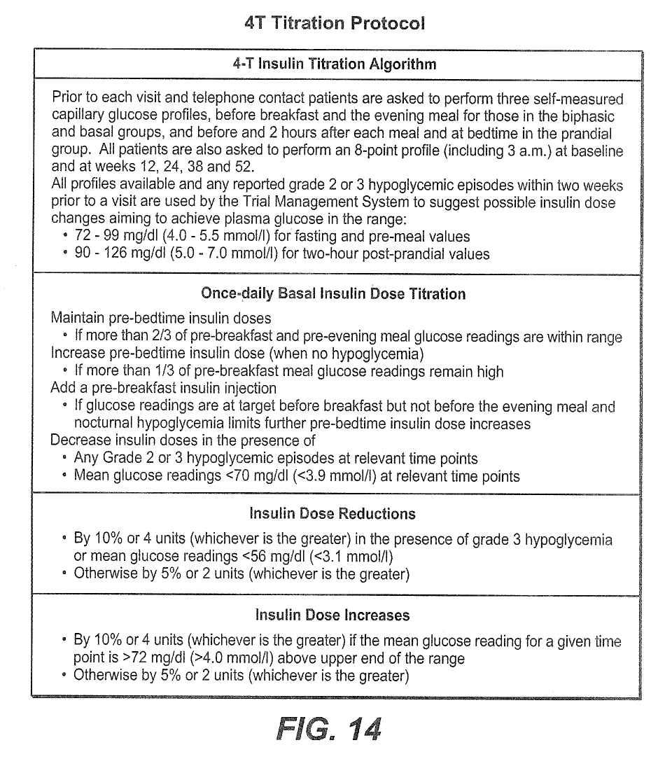

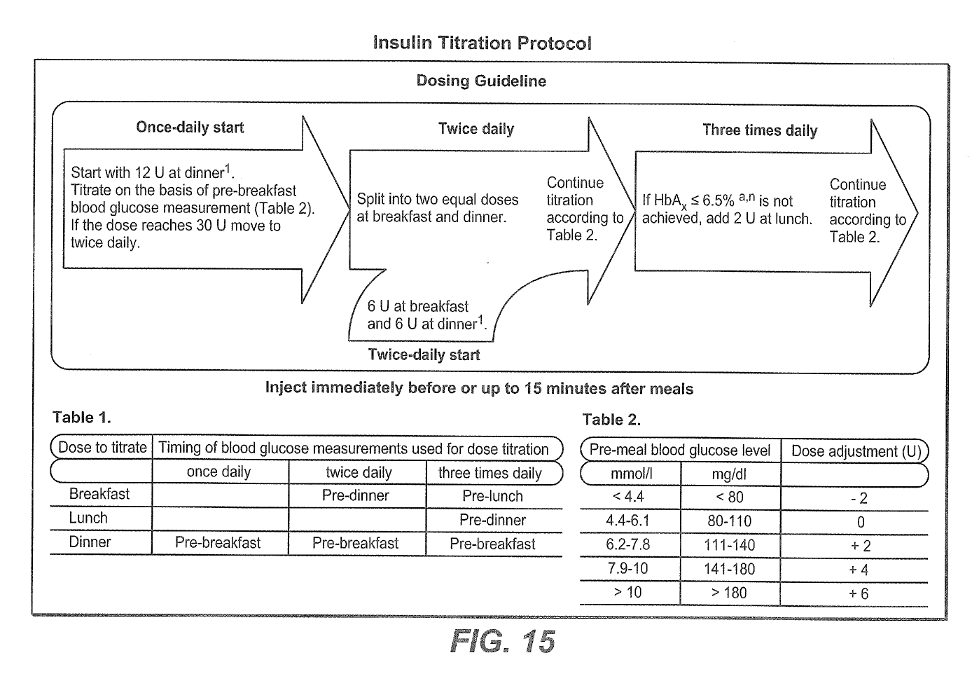

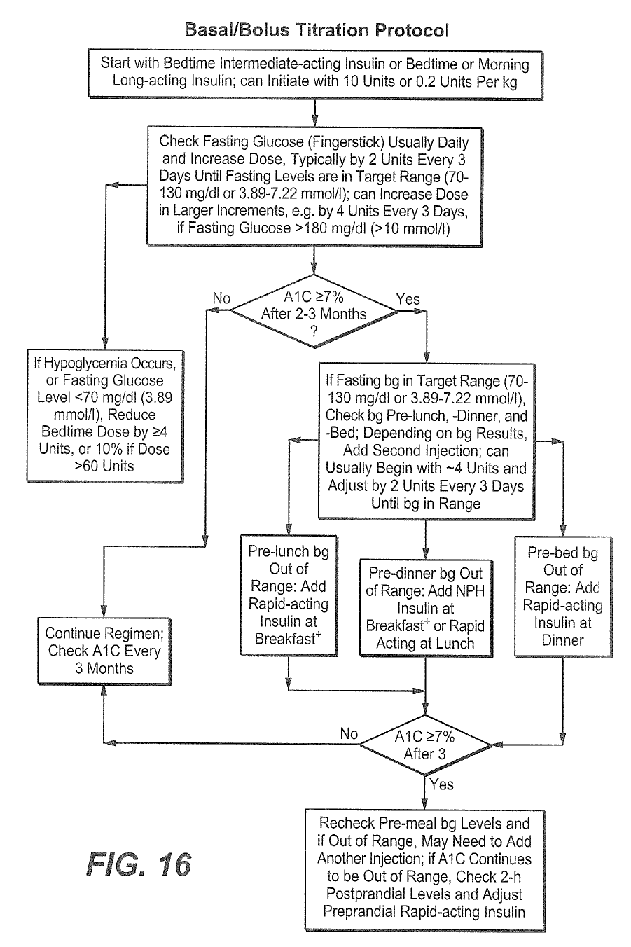

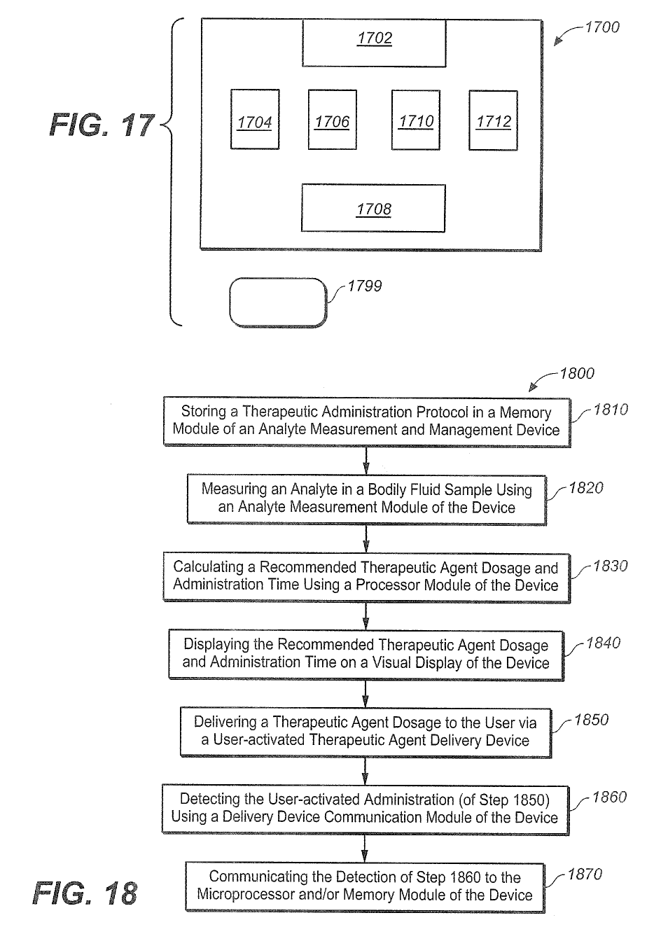

Referring to FIG. 1, a diabetes management system can be provided that includes at least two components. The first component can include analyte measurement device 100 that has a housing 101, a processor 1706 and memory 1704 (shown schematically in FIG. 17) disposed in the housing 101. The first component can include measurement unit 1702 (shown in FIG. 17) in communication with the processor 1706 to provide a numerical value representing generally an amount of analyte in body fluids to the processor 1706 so that this numerical value can be utilized to provide dosage recommendation for delivery of therapeutic agent according to one of a plurality of recommended administration protocols. The device 100 includes a display 104 in electrical communication with the processor to display information relating to analyte and the therapeutic agent. The device includes first communication module 1710 (FIG. 17) preferably a first wireless module coupled to the processor and memory to store data received by the first wireless module in the memory 1704. The second component of the diabetes management system can include a therapeutic agent delivery device 12, which has a housing, preferably elongated and of sufficient size to be handled by a human hand comfortably. The device 12 is provided with electronic module 121 to record dosage amount delivered by the user. The device 12 may include a second wireless module disposed in the housing that, automatically without prompting from a user, transmits a signal to the first wireless module. The signal can include data to (a) type of therapeutic agent delivered; and (b) amount of therapeutic agent delivered to the user; or (c) type of therapeutic agent device from which the therapeutic agent was administered. An additional component, which can also be utilized, with the first and second component is a health care provider computer 13 which can be used to communicate with the analyte measurement device or the delivery device. In one example, the computer 13 can be connected via a mobile network to the device 100 or 13. Alternatively, the computer 13 can be connected for communication via a short-range wireless network such as, for example, infrared, Bluetooth or WiFi. In the system shown exemplarily, the computer 13 can be located remotely in a diabetes clinic or hospital so that certain therapeutic protocols, which have been customized for a particular diabetic user's physiological requirements, can be transferred to such user remotely. The therapeutic protocol may include, for example, a 4T-Titration Protocol (FIG. 14), multiple daily injection titration protocol (FIG. 15), Basal/Bolus Titration Protocol (FIG. 16), or any other suitable protocol. The physiological requirements may include, for example, height, weight, insulin resistance, health profiles and any other physiological datum of the user relevant in the treatment of diabetic user.

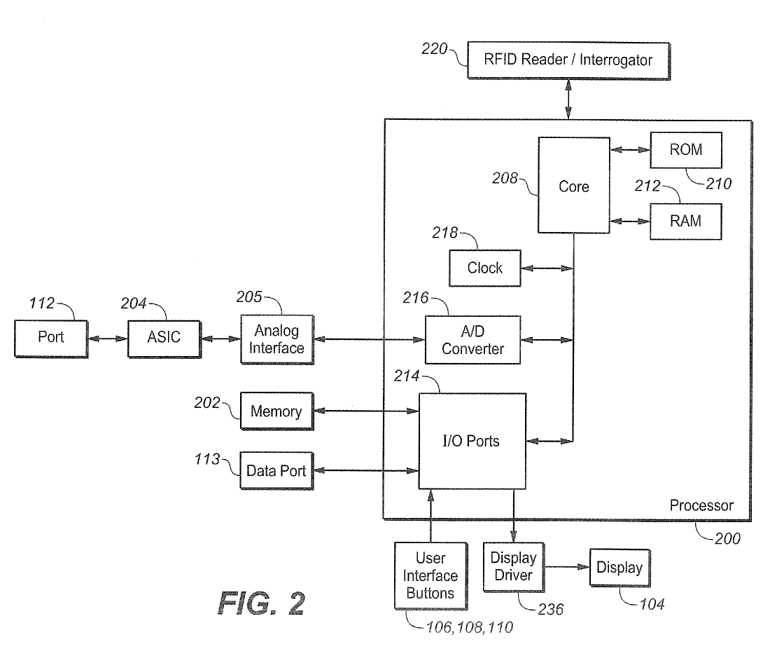

Referring to FIG. 2, an exemplary internal layout of analyte measurement device 100 is shown. Analyte measurement device 100 may include a processor 200, which in some embodiments described and illustrated herein is a 32-bit RISC microcontroller. The processor can be bi-directionally connected via I/O ports 214 to memory 202, which in some embodiments described and illustrated herein is an EEPROM. Also connected to processor 200 via I/O ports 214 are the data port 113, the user interface buttons 106, 108, and 110, and a display driver 236. Data port 113 can be connected to processor 200, thereby enabling transfer of data between memory 202 and an external device, such as a personal computer. User interface buttons 106, 108, and 110 are directly connected to processor 200. Processor 200 controls display 104 via display driver 236.

In the preferred embodiments, analyte measurement device 100 may include an Application Specific Integrated Circuit (ASIC) 204, providing electronic circuitry used in measurements of glucose level in blood that has been applied to a test strip 10 inserted into strip port 112. Analog voltages can pass to and from ASIC 204 by way of analog interface 205. Analog signals from analog interface 205 can be converted to digital signals by A/D converter 216. Processor 200 may further include core 208, ROM 210 (containing computer code), RAM 212, and clock 218. Additionally, the processor 200 is configured (or programmed) to disable all of the user interface buttons except for a single button upon a display of an analyte value by the display unit such as, for example, during a time period after an analyte measurement. In an alternative embodiment, the processor 200 is configured (or programmed) to ignore any input from all of the user interface buttons except for a single button upon a display of an analyte value by the display unit.

In the preferred embodiments, analyte measurement device 100 may include a Radio Frequency Identification (RFID) Reader/Interrogator 220. Additionally, the reader/interrogator communicates with a passive RFID tag to identify the therapeutic agent delivery device. In an alternative embodiment the reader/interrogator communicates with a passive RFID tag within the therapeutic agent delivery device to detect administration of the therapeutic agent.

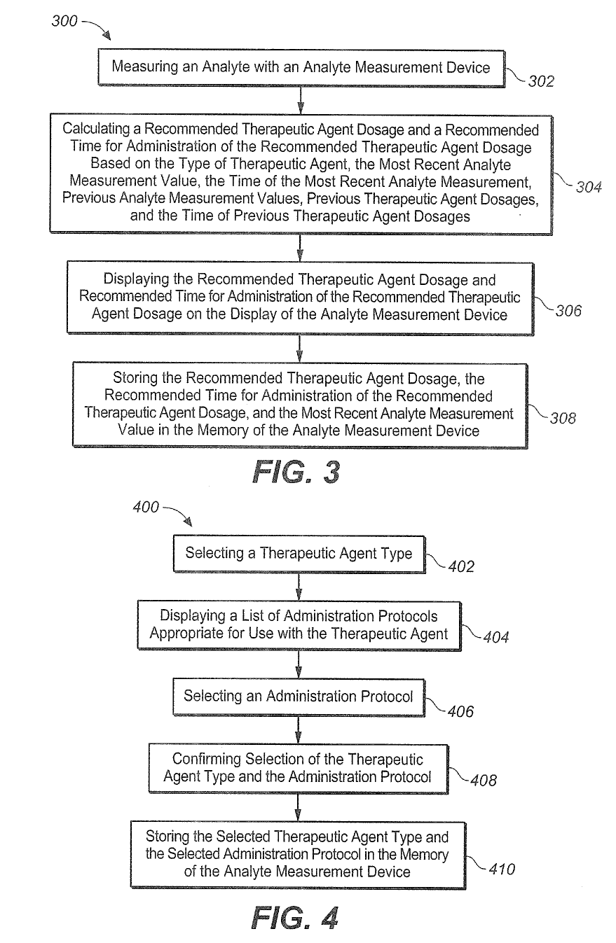

FIG. 3 illustrates an exemplary flow chart illustrating a method of operating an analyte measurement device. Method 300 comprises steps 302, 304, 306, and 308. Step 302 includes measuring an analyte with the analyte measurement device. In these embodiments, the analyte is measured using electrochemical techniques and the analyte is blood glucose. In other embodiments described and illustrated herein the analyte is measured photo metrically, and the analyte is blood glucose. In other embodiments described and illustrated herein the analyte is measured using immunoassay or electrochemical techniques. Step 304 includes calculating a recommended therapeutic agent dosage and a recommended time for administration of the recommended therapeutic agent dosage based on the type of therapeutic agent, the most recent analyte measurement value, the time of the most recent analyte measurement, previous analyte measurement values, previous therapeutic agent dosages, and the time of previous therapeutic agent dosages. Additionally, the therapeutic agent is insulin and the analyte measurement is blood glucose. Step 306 includes displaying the recommended therapeutic agent dosage and recommended time for administration of the recommended therapeutic agent dosage on the display of the analyte measurement device. Further, the display of the analyte measurement device is an LCD, and the recommended therapeutic agent dosage and recommended time for administration of the recommended therapeutic agent dosage is displayed on a user interface. Step 308 includes storing the recommended therapeutic agent dosage, the recommended time for administration of the recommended therapeutic agent dosage, and the most recent analyte measurement value in the memory of the analyte measurement device. Additionally, the memory of the analyte measurement device includes a removable memory such as a single in-line memory module (SIMM) card. Additionally, the method may further include displaying a reminder to administer the recommended therapeutic agent dosage on the display of the analyte measurement device. Additionally, reminders can be audible, such as a beep, or sensory, such as vibration. Additionally, the method may further include displaying a reminder to measure an analyte on the display of the analyte measurement device. Additionally, the reminder reminds the user to measure their blood glucose. Additionally, the reminder reminds the user to administer insulin. Further, the method may further include determining if the current time and date fall within a time window for analyte measurement; emitting an alarm if analyte measurement has not occurred within the time window; and storing a record of the alarm in the memory of the analyte measurement device. Further, the current date and time is determined using the analyte measurement device internal clock. In these embodiments, the method may further include retrieving the recommended therapeutic agent dosage from the memory of the analyte measurement device; determining if the current time and date fall within a time window for administration of the recommended therapeutic agent dosage; emitting an alarm if administration of the recommended therapeutic agent dosage has not occurred within the time window for administration of the recommended therapeutic agent dosage; and storing a record of the alarm in the memory of the analyte measurement device. Further, alarm records stored in the memory of the analyte measurement device can be used to establish compliance with recommended measurement and therapeutic agent administration protocols.

In these embodiments, the therapeutic agent is long acting insulin and the time window is in the early morning upon awakening or the late evening before bedtime. Further, the therapeutic agents include both long acting and rapid acting insulins and the time window for administering the long acting insulin is in the early morning or the late evening and the time window for administering the rapid acting insulin is premeal. Additionally, the therapeutic agent is one of an oral antidiabetic agent, a GLP-1 agent, insulin and insulin mixes, or a combination thereof. Further, the therapeutic agent is medication for metabolic management, hormonal therapies, oncology, pain management, regenerative medicine, or a combination thereof. Further, the therapeutic agent is a medication used in the management of diabetes.

In these embodiments, the analyte measurement device automatically displays the recommended therapeutic agent dosage after taking a blood glucose measurement, or after turning the analyte measurement device on. Additionally, the recommended therapeutic agent dosage can be a function of at least one previous analyte measurement value if the measurement analyte value is greater than or less than preset thresholds. For example, if a blood glucose measurement is high the recommended insulin dosage may be increased, whereas if a blood glucose measurement is low the insulin dosage may be decreased. Further, the analyte measuring device queries a user and upon user acceptance displays the recommended therapeutic agent dosage. The query can be in the form of a user interface prompt displayed on the analyte measurement device. User acceptance can include pressing a specific user interface button. Further, the recommended therapeutic agent dosage is displayed in the form of units of insulin.

In these embodiments, the recommended therapeutic agent dosage is displayed to a user in the format of user button pushes on the associated user-activated therapeutic agent delivery device. For example, such button pushes can be used to actuate the delivery of a predetermined amount of therapeutic agent by displacement from the user-activated therapeutic agent delivery device. A non-limiting example of such a user-activated therapeutic delivery device is described in the aforementioned U.S. Provisional Patent Application No. 61/040,024 (tentatively identified by Attorney Docket No. LFS-5180).

In these embodiments, a user can toggle between displaying the recommended therapeutic agent dosage in the form of insulin units or button pushes. Toggling between insulin units and button pushes can be accomplished by way of the analyte measurement device user interface. Additionally, the recommended therapeutic agent dosage is displayed in graphical form. Graphical forms can include column, bar, line, pie, circles, and lights. Further, the recommended therapeutic agent dosage is presented to a user in audio form by an audio module of the testing device. Further, the recommended therapeutic agent dosage does not exceed a preset maximum daily dosage. For example, a maximum daily dosage of insulin may be entered into the analyte measurement device, and subsequently limit the daily recommended therapeutic agent dosage. Additionally, a time stamp for the analyte measurement is used to determine if the measurement is pre-breakfast, pre-lunch, pre-dinner, or pre-snack. For example, if the analyte measurement is performed at 7:00 am, it could be considered to be pre-breakfast, while a test performed at 5:00 pm could be considered to be pre-dinner. Further, the method may further include prompting a user to confirm that the measurement is pre-breakfast, pre-lunch, pre-dinner, or pre-snack. Further, the method may further include prompting a user to confirm that the measurement is pre-breakfast, pre-lunch, pre-dinner, or pre-snack; and, prompting the user to enter a start time of the most recent meal or snack if the meal was not pre-breakfast, pre-lunch, or pre-dinner. For example, if a measurement occurs outside the preset windows for breakfast, lunch, dinner, than the specific start time of a snack can be entered.

In these embodiments, the method may further include retrieving the recommended therapeutic agent dosage from the memory of the analyte measurement device; displaying the recommended therapeutic agent dosage and the recommended time for administration of the recommended therapeutic agent dosage on the display of the analyte measurement device; prompting a user to confirm administration of the recommended therapeutic agent if the current time and date is approximately equal to the recommended time for administration of the recommended therapeutic agent dosage; pressing at least one of the user interface buttons to confirm administration of therapeutic agent; and storing a record of the administration of therapeutic agent in the memory of the analyte measurement device. Further, the analyte measurement device is a blood glucose meter, the therapeutic agent is insulin, the administration is performed with an insulin dosage device, and the dosage is confirmed by pressing a user interface button on the blood glucose meter. Further, the method may further include prompting a user to enter the amount of therapeutic agent administered if the amount of therapeutic agent administered differs from the recommended therapeutic agent dosage. For example, if the recommended dosage is 4 units and only 3 units are injected, than the user would enter 3 units. Further, the method may further include prompting a user to enter the amount of therapeutic agent administered if the amount of therapeutic agent administered differs from the recommended therapeutic agent dosage; and, prompting the user to confirm the amount of therapeutic agent administered. Confirming the actual dosage increases the accuracy of dosage recommendations. Further, the method may further include prompting a user to enter the amount of therapeutic agent administered if the amount of therapeutic agent administered differs from the recommended therapeutic agent dosage; prompting the user to confirm the amount of therapeutic agent administered; and, storing the amount of therapeutic agent administered in the memory of the analyte measurement device. As mentioned previously, the memory of the analyte measurement device may include a removable portion, such as a SIMM card. Further, the method may further include activating a reporting summary; calculating the percentage of actual versus recommended analyte measurements and the percentage of actual versus recommended therapeutic agent dosages; and displaying the percentages. Reporting summaries are useful in accessing conformance to recommended protocols, and are particularly useful in communicating with health care practitioners. Further, the method may further include calculating and displaying an analyte measurement average for a weekly, monthly, quarterly, yearly, or 6 week time period. Further, the method may further include calculating a percentage of out-of-range high and out-of-range low analyte measurements over a period of time, and displaying the percentage of out-of-range high and out-of-range low analyte measurements and time period. High and low ranges can be preset on the measurement device or set by the user or a health care practitioner, and are useful in managing conditions such as diabetes. Further, the method may further include activating a reporting summary; calculating the percentage of actual versus recommended analyte measurements and the percentage of actual versus recommended therapeutic agent dosages over a period of time; and displaying the percentages and period of time. Additionally, the method may further include activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. External devices include personal computers, network computer systems, external removable memory readers, PDAs, and mobile phones. Further, the method may further include uploading the downloaded data into a database linked to insurance incentives, disease management, or motivational programs. Further, the method may further include uploading the downloaded data into a database linked to pay-for-performance programs. Further, insurance incentives, motivational programs, and pay-for-performance programs can be accessed via the Internet. Further, the method may further include uploading the downloaded data into a database linked to clinical data registries.

In these embodiments, the method may further include receiving at least one signal from a dosage device confirming administration of therapeutic agent; and storing a record of the administration of therapeutic agent in the memory of the analyte measurement device. Furthermore, methods according to the present invention can include steps of retrieving a recommended therapeutic agent dosage and associated recommended administration time from the memory (also referred to herein as a memory module), and displaying such a retrieved recommended therapeutic agent dosage and administration time to user on the visual display of the analyte measurement device. Additionally, the signal is a wireless signal such as Bluetooth or radio-frequency identification (RFID). Further, the dosage device is a pump or a pen. Further, the RFID component in the dosage device is passive and the RFID component in the analyte measurement device is active. Further, the RFID component in the dosage device is powered by receiving signals from the analyte measurement device. Further, the dosage device includes a passive, active, or semi-passive radio-frequency tag. Additionally, the method may further include storing the amount of therapeutic agent remaining in the dosage device in the memory of the analyte measurement device. Additionally, the method may further include alerting a user if the amount of therapeutic agent remaining in the dosage device is less than the amount required for a preset number of dosages or expected daily dosage. Further, the method may further include displaying the amount of therapeutic agent remaining in the dosage device in the form of units, days, or graphs. Further, the signal can include information related to therapeutic agent type, cartridge type, cartridge volume, and type of dosage device. For example, an insulin pump could send a signal to the analyte measurement device that includes information in respect to type of insulin being used, the type of pump cartridge, the volume of the pump cartridge, the type of pump, and the associated bolus increment per button push (for example 1 button push is equivalent to 3 units). Additionally, the method may further include of using the associated bolus increment per button push as input into the protocol algorithm. Additionally, the method may further include displaying the amount of therapeutic agent remaining in the dosage device after receiving the signal. Additionally, the method may further include of displaying the remaining number of button pushes necessary to complete the recommended dosage. Additionally, the method may further include sending a signal from the analyte measurement device to the dosage device to lock down the dosage device if the amount of therapeutic agent delivered exceeds a preset maximum for a preset time window. For example, if the daily maximum dosage is exceeded, a signal can be sent from the analyte measurement device to the pump to stop delivering insulin until the next day. Additionally, the method may further include sending a signal from the analyte measurement device to multiple dosage devices to stop delivering therapeutic agent if the amount of therapeutic agent delivered exceeds a preset maximum for a preset time window. Additionally, the analyte measurement device can determine which form of therapeutic agent dosage units to display based upon the signal from the dosage device. Further, the analyte measurement device can provide an alarm if a signal is received from a dosage device outside a preset time window. Further, the method may further include activating a reporting summary; calculating the percentage of actual versus recommended analyte measurements and the percentage of actual versus recommended therapeutic agent dosages; and displaying the percentages. Additionally, the method may further include calculating and displaying an analyte measurement average for a weekly, monthly, quarterly, yearly, or 6 week time period. Additionally, the method may further include calculating a percentage of out-of-range high and out-of-range low analyte measurements over a period of time; and, displaying the percentage of out-of-range high and out-of-range low analyte measurements and time period. Further, the method may further include activating a reporting summary; calculating the percentage of actual versus recommended analyte measurements and the percentage of actual versus recommended therapeutic agent dosages over a period of time; and displaying the percentages and period of time. Further, the method may further include activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. Additionally, the method may further include uploading the downloaded data into a database linked to insurance incentives, disease management, or motivational programs. Additionally, the method may further include uploading the downloaded data into a database linked to pay-for-performance programs. Additionally, the method may further include uploading the downloaded data into a database linked to clinical data registries.

FIG. 4 illustrates an exemplary flow chart illustrating a method of operating an analyte measurement device. Method 400 may include steps 402, 404, 406, 408, and 410. Step 402 includes selecting a therapeutic agent type. In these embodiments, step 402 includes selecting a type of insulin. Step 404 includes displaying a list of administration protocols appropriate for use with the therapeutic agent. In these embodiments, step 404 includes displaying a list of administration protocols that include measurement frequency, dosage frequency, and dosage amounts. Step 406 includes selecting an administration protocol. Step 408 includes confirming selection of the therapeutic agent type and the administration protocol. Step 410 includes storing the selected therapeutic agent type and the selected administration protocol in the memory of the analyte measurement device. In these embodiments, the administration protocol is selected by way of a user interface menu. In these embodiments, selecting the administration protocol includes entering a passcode, preventing inadvertent changes to the administration protocol. In these embodiments, selecting a therapeutic agent type is initiated by inserting a hardware key into the analyte measurement device. In these embodiments, a hardware key is inserted into the strip port connector or the data port to initiate selection of a therapeutic agent type. In these embodiments, selecting a therapeutic agent type and administration protocol is initiated as a result of an analyte value such as an HbA1c value being in a preset range or a series of analyte measurement values, such as blood glucose values, being in a preset range.

In these embodiments, the administration protocol may include one or more initiation, titration, and testing regimens. In these embodiments, the method may further include selecting a time zone on the analyte measurement device. In these embodiments, the method may further include confirming a recommended not-to-exceed daily dosage of therapeutic agent. In these embodiments, the method may further include entering a time zone and approximate time windows for meals, snacks, wake-up, and bedtime; and, storing the time zone and approximate time windows for meals, snacks, wake-up, and bedtime in the memory of the analyte measurement device. In these embodiments, the method may further include accepting or modifying the time zone and approximate time windows for meals, snacks, wake-up, and bedtime; and, storing the time zone and approximate time windows for meals, snacks, wake-up, and bedtime in the memory of the analyte measurement device. In these embodiments, the method may further include initiating an administration protocol updating function, downloading an updated administration protocol; confirming completion of the download, selecting the updated administration protocol, displaying a summary of the updated administration protocol, and storing the updated administration protocol in the memory of the analyte measurement device. Updates ensure the use of the most up-to-date protocols and regimens. In these embodiments, the downloading can occur wirelessly, through a USB or other physical connection, or through connection to a removable memory card inserted into the analyte measurement device. In these embodiments, the analyte measurement device can be linked electronically to a network computer and be identified by a software code unique to the analyte measurement device. In these embodiments, initiating administration protocol updating occurs automatically or when activated by a user. For example, updating can occur automatically when connecting the analyte measurement device to a network, or can be manually activated by way of the user interface. In these embodiments, a user confirms initiation of the administration protocol updating function.

In these embodiments, the method may further include activating a reporting summary; calculating the percentage of actual versus recommended analyte measurements and the percentage of actual versus recommended therapeutic agent dosages; and displaying the percentages. In these embodiments, the method may further include calculating and displaying an analyte measurement average for a weekly, monthly, quarterly, yearly, or 6 week time period. In these embodiments, the method may further include calculating a percentage of out-of-range high and out-of-range low analyte measurements over a period of time; and, displaying the percentage of out-of-range high and out-of-range low analyte measurements and time period.

In these embodiments, the method may further include activating a reporting summary; calculating the percentage of actual versus recommended analyte measurements and the percentage of actual versus recommended therapeutic agent dosages over a period of time; and displaying the percentages and period of time. In these embodiments, the method may further include activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. In these embodiments, the method may further include uploading the downloaded data into a database linked to insurance incentives, disease management or motivational programs. In these embodiments, the method may further include uploading the downloaded data into a database linked to pay-for-performance programs. In these embodiments, the method may further include uploading the downloaded data into a database linked to clinical data registries.

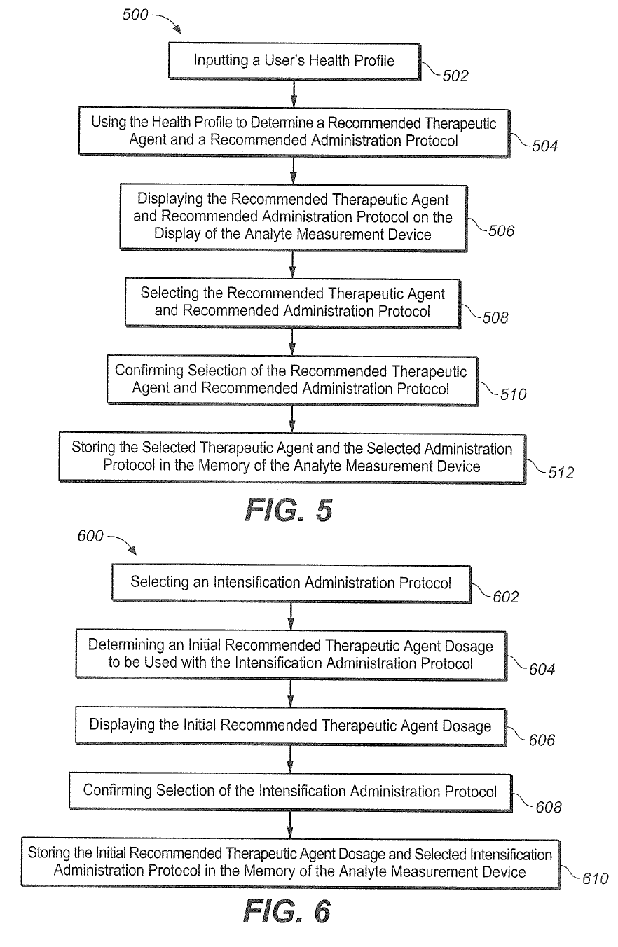

FIG. 5 illustrates an exemplary flow chart illustrating a method of operating an analyte measurement device. Method 500 may include steps 502, 504, 506, 508, 510, and 512. Step 502 includes inputting a user's health profile. Step 504 includes using the health profile to determine a recommended therapeutic agent and a recommended administration protocol. Step 506 includes displaying the recommended therapeutic agent and recommended administration protocol on the display of the analyte measurement device. Step 508 includes selecting the recommended therapeutic agent and recommended administration protocol. Step 510 includes confirming selection of the recommended therapeutic agent and recommended administration protocol. Step 512 includes storing the selected therapeutic agent and the selected administration protocol in the memory of the analyte measurement device. In these embodiments, the user's health profile includes lifestyle and eating habits information. In these embodiments, the user's health profile includes the largest meal size the patient consumes. In these embodiments, the user's health profile can include previous blood glucose results, hemoglobin A1C results, weight, fasting glucose, or the user's tolerance to glucose. In these embodiments, the method may further include customizing the recommended administration protocol by setting an analyte measurement frequency or adjusting the therapeutic agent dosing. In these embodiments, the method may further include measuring an analyte with the analyte measurement device; calculating a starting therapeutic agent dosage based on the user's weight and the therapeutic agent initiation dosage multiplier; displaying the recommended starting therapeutic agent dosage and recommended time for therapeutic agent administration on the display of the analyte measurement device; and storing the recommended therapeutic agent dosage, the recommended time for therapeutic agent administration, and the current analyte measurement value into the memory of the analyte measurement device. If desired, methods and devices according to embodiments described and illustrated herein can be configured to allow user confirmation, customization and/or acceptance of protocols and any recommendations thereof. In these embodiments, the method may further include initiating an administration protocol updating function; downloading an updated administration protocol, confirming completion of the download, selecting the updated administration protocol, displaying a summary of the updated administration protocol, and storing the updated administration protocol in the memory of the analyte measurement device. In these embodiments, the downloading can occur wirelessly, through a USB or other physical connection, or through connection to a memory card inserted into the analyte measurement device. In these embodiments, the analyte measurement device can be linked electronically to a network computer and be identified by a software code unique to the analyte measurement device. In these embodiments, initiating administration protocol updating occurs automatically or when activated by a user. In these embodiments, a user confirms initiation of the administration protocol updating function. In these embodiments, the method may further include activating a reporting summary, calculating a percentage of actual versus recommended analyte measurements and a percentage of actual versus recommended therapeutic agent dosages; and displaying the percentages.

In these embodiments, the method may further include calculating and displaying an analyte measurement average for a weekly, monthly, quarterly, yearly, or 6 week time period. In these embodiments, the method may further include calculating a percentage of out-of-range high and out-of-range low analyte measurements over a period of time; and, displaying the percentage of out-of-range high and out-of-range low analyte measurements and time period. In these embodiments, the method may further include activating a reporting summary, calculating the percentage of actual versus recommended analyte measurements and the percentage of actual versus recommended therapeutic agent dosages over a period of time; and displaying the percentages and period of time. In these embodiments, the method may further include activating a reporting summary, calculating a percentage of actual versus recommended analyte measurements and a percentage of actual versus recommended therapeutic agent dosages, activating a downloading function; downloading data and reports from the analyte measurement device, confirming completion of the download, and storing the downloaded data and reports in the memory of an external device. In these embodiments, the method may further include uploading the downloaded data into a database linked to insurance incentives, disease management or motivational programs. In these embodiments, the method may further include uploading the downloaded data into a database linked to pay-for-performance programs. In these embodiments, the method may further include uploading the downloaded data into a database linked to clinical data registries.

FIG. 6 illustrates an exemplary flow chart illustrating a method of operating an analyte measurement device. Method 600 may include steps 602, 604, 606, 608, and 610. Step 602 includes selecting an intensification administration protocol. Step 604 includes determining an initial recommended therapeutic agent dosage to be used with the intensification administration protocol. Step 606 includes displaying the initial recommended therapeutic agent dosage. Step 608 includes confirming selection of the intensification administration protocol. Step 610 includes storing the initial recommended therapeutic agent dosage and selected intensification administration protocol in the memory of the analyte measurement device. In these embodiments, the intensification administration protocol is suggested after inputting the user's existing administration protocol. In these embodiments, the intensification administration protocol is automatically suggested by the analyte measurement device if analyte measurements are high. In these embodiments, the intensification administration protocol includes the use of short acting and long acting insulin.

In these embodiments, the intensification administration protocol includes switching from long acting insulin to premixed insulin. In these embodiments, the intensification administration protocol includes switching from premixed insulin to short acting insulin and long acting insulin. In these embodiments, the intensification administration protocol includes switching from one therapeutic agent to another. In these embodiments, the intensification administration protocol includes the use of one or more therapeutic agents. In these embodiments, the method may further include notifying the user that a new intensification administration protocol has been implemented; and, displaying times to conduct analyte measurements, times to administer therapeutic agent, and type of therapeutic agent to administer. In these embodiments, the method may further include querying the user as to whether reminders or alarms should be displayed if analyte testing or therapeutic agent administration does not occur as specified in the intensification administration protocol. In these embodiments, the method may further include displaying post-meal analyte measurement reminders at 1, 2, 3, and 4 hours after meals. In these embodiments, reminders or alarms can be automatically or manually disabled. In these embodiments, the method may further include displaying a report summarizing the data related to the intensification administration protocol and at least one previous administration protocol.

In these embodiments, the method may further include initiating an intensification administration protocol updating function, downloading an updated intensification administration protocol, confirming completion of the download, selecting the updated intensification administration protocol, displaying a summary of the updated intensification administration protocol, and storing the updated intensification administration protocol in the memory of the analyte measurement device. In these embodiments, the downloading can occur wirelessly, through a USB or other physical connection, or through connection to a memory card inserted into the analyte measurement device. In these embodiments, the analyte measurement device can be linked electronically to a network computer and be identified by a software code unique to the analyte measurement device. In these embodiments, initiating administration protocol updating occurs automatically or when activated by a user. In these embodiments, a user confirms initiation of the administration protocol updating function. In these embodiments, the method may further include activating a reporting summary function of the device, calculating a percentage of actual versus recommended analyte measurements and a percentage of actual versus recommended therapeutic agent dosages, and calculating average premeal and 2 hr postmeal analyte values by mealtime (like breakfast, lunch and dinner).

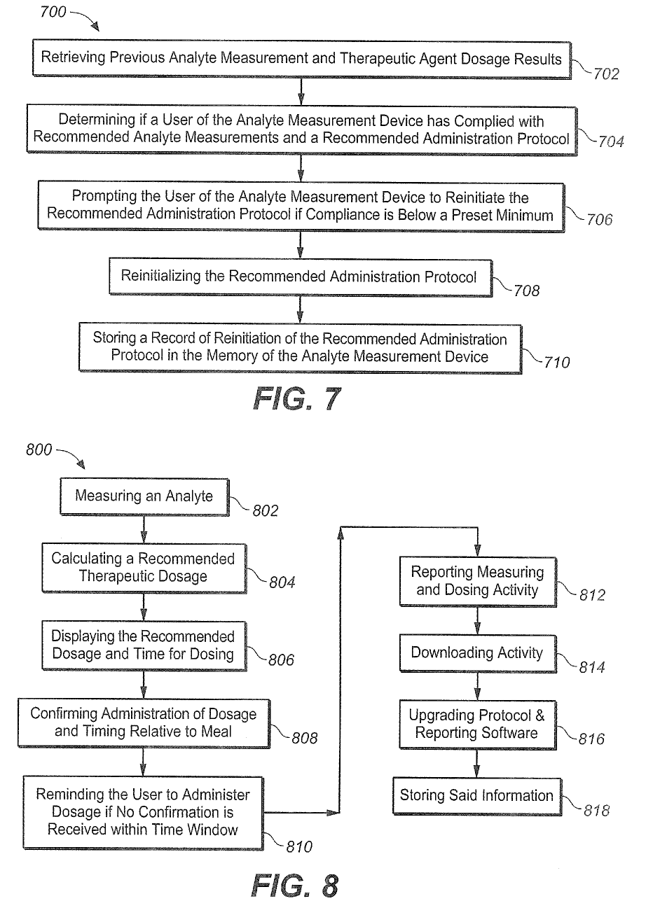

FIG. 7 illustrates an exemplary flow chart illustrating a method of operating an analyte measurement device. Method 700 may include steps 702, 704, 706, 708, and 710. Step 702 includes retrieving previous analyte measurement and therapeutic agent dosage results. In these embodiments, previous analyte measurement and therapeutic agent dosage results are retrieved from the analyte measurement device's memory, or from a removable memory that is coupled with the analyte measurement device. Step 704 includes determining if a user of the analyte measurement device has complied with recommended analyte measurements and a recommended administration protocol. Compliance may include making analyte measurements and therapeutic agent dosages within specified time windows. Step 706 includes prompting the user of the analyte measurement device to reinitiate the recommended administration protocol if compliance is below a preset minimum.

Step 708 includes reinitializing the recommended administration protocol. Step 710 includes storing a record of reinitiation of the recommended administration protocol in the memory of the analyte measurement device. In these embodiments, the method may further include prompting the user to enter a reason for noncompliance. In these embodiments, the method may further include suggesting to the user that they contact a healthcare provider prior to reinitializing the recommended administration protocol if the reason for noncompliance is illness. In these embodiments, a healthcare provider can preset compliance limits. In these embodiments, the analyte measurement device can automatically reinitialize the recommended administration protocol if the user is noncompliant in respect to analyte measurements or therapeutic agent dosages. In these embodiments, the analyte measurement device can automatically reinitialize the recommended administration protocol if the user was noncompliant for a preset time period.

In these embodiments, the analyte measurement device can automatically continue the recommended administration protocol if the user was noncompliant for less than a preset time period. In these embodiments, the analyte measurement device can automatically disable the recommended administration protocol upon noncompliance. In these embodiments, the recommended administration protocol can be restarted. In these embodiments, the method may further include sending an alert to a health care practitioner that non-compliance has occurred.

FIG. 8 illustrates an exemplary flow chart illustrating a method of operating an analyte measurement device. Method 800 may include steps 802, 804, 806, 808, 810, 812, 814, 816, and 818. Step 802 includes measuring an analyte with an analyte measurement device. Step 804 includes calculating a recommended therapeutic dosage. Step 806 includes displaying the recommended dosage and time for dosing. Step 808 includes confirming administration of dosage and timing relative to a meal. Step 810 includes reminding the user to administer dosage if no confirmation is received within a time window. Step 812 includes reporting measuring and dosing activity. Step 814 includes downloading measurement and dosing activity. Step 816 includes upgrading the protocol & reporting software. Step 818 includes storing measurement, dosage, and reporting information in the memory of the analyte measurement device.

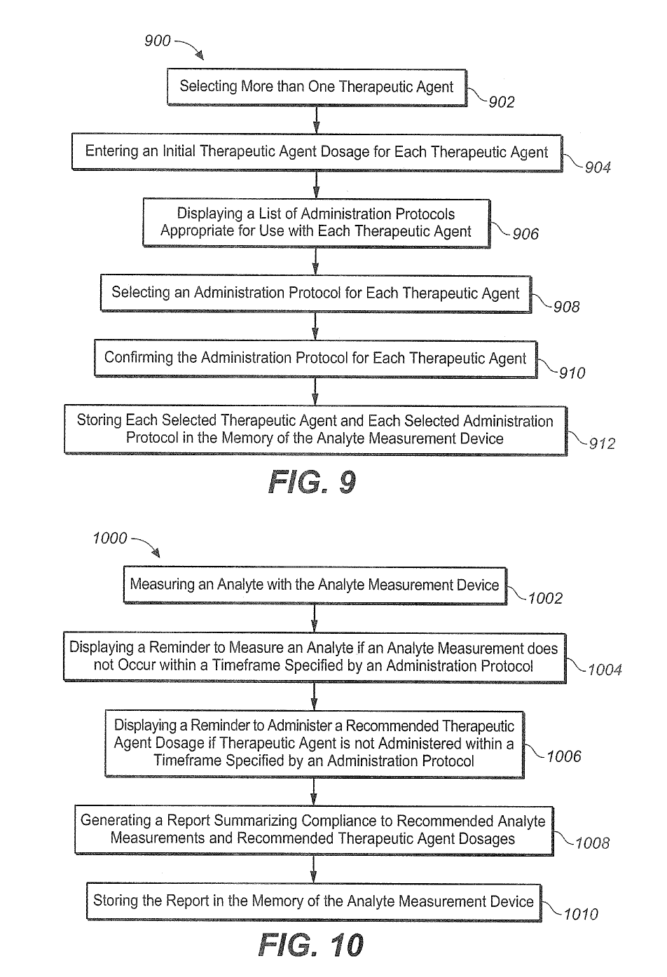

FIG. 9 illustrates an exemplary flow chart illustrating a method of operating an analyte measurement device. Method 900 may include steps 902, 904, 906, 908, 910, and 912. Step 902 includes selecting more than one therapeutic agent. Step 904 includes entering an initial therapeutic agent dosage for each therapeutic agent. Step 906 includes displaying a list of administration protocols appropriate for use with each therapeutic agent. Step 908 includes selecting an administration protocol for each therapeutic agent. Step 910 includes confirming the administration protocol for each therapeutic agent. Step 912 includes storing each selected therapeutic agent and each selected administration protocol in the memory of the analyte measurement device. In these embodiments, the administration protocol includes recommended times for analyte measurement. In these embodiments, the therapeutic agents may include oral antidiabetics, GLP-1 analogues, insulin, or metabolic agents. In these embodiments, the method may further include prompting the user to activate measurement and dosage reminders should measurements or dosages occur outside a specified window of time.

FIG. 10 illustrates an exemplary flow chart illustrating a method of operating an analyte measurement device. Method 1000 may include steps 1002, 1004, 1006, 1008, and 1010. Step 1002 includes measuring an analyte with the analyte measurement device. Step 1004 includes displaying a reminder to measure an analyte if an analyte measurement does not occur within a timeframe specified by an administration protocol. Step 1006 includes displaying a reminder to administer a recommended therapeutic agent dosage if therapeutic agent is not administered within a timeframe specified by an administration protocol. Step 1008 includes generating a report summarizing compliance to recommended analyte measurements and recommended therapeutic agent dosages. Step 1010 includes storing the report in the memory of the analyte measurement device. In these embodiments, confirmation of a recommended therapeutic agent dosage occurs manually or automatically. In these embodiments, confirmation of a recommended therapeutic agent dosage occurs automatically. Additionally, the recommended therapeutic agent dosage is administered with a pillbox, a user-activated insulin pen, a user-activated inhaler, or user-activated pump. Further, the pillbox, insulin pen, inhaler, or pump sends an RFID signal to the analyte measurement device automatically confirming delivery of the recommended therapeutic agent dosage.

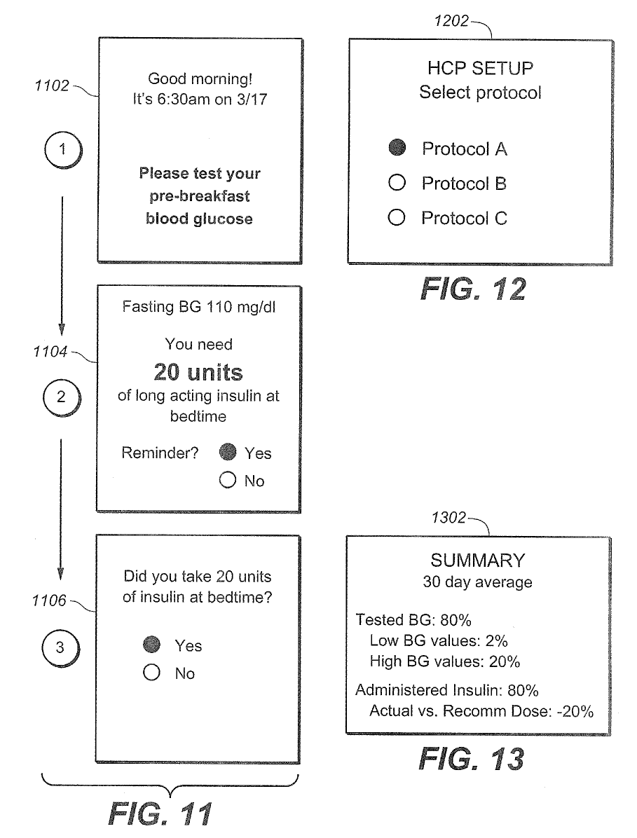

FIG. 11 illustrates a series of user interface screens displayed during a method of operating an analyte measurement device. In screen 1102, the user is prompted to measure their pre-breakfast blood glucose. Screen 1104 displays the measured pre-breakfast (or fasting) blood glucose result, a recommended dose of insulin and its time of administration. The user is also prompted to set a reminder. Screen 1106 illustrates the reminder, displayed just before the recommended administration time.

FIG. 12 illustrates a user interface screen displayed during a method of operating an analyte measurement device. In screen 1202, a health care practitioner or user is prompted to selects an insulin administration protocol.

FIG. 13 illustrates a user interface screen that displayed during a method of operating an analyte measurement device. In screen 1302, a compliance summary of analyte measurement and therapeutic agent dosing over a certain time period is displayed.

FIG. 14 illustrates an exemplary treat-to-target protocol identified as “4T-Titration Protocol” that could be used as a therapeutic protocol. FIG. 15 illustrates an exemplary treat-to-target insulin protocol that could be used as yet another therapeutic protocol. FIG. 16 illustrates an exemplary treat-to-target intensification protocol identified as “Basal/Bolus Titration Protocol” that could be used as a further therapeutic protocol.

FIG. 17 is a simplified block diagram of an analyte measurement and management device 1700 for use with a user-activated therapeutic agent delivery device 1799 according to an embodiment described and illustrated herein. An analyte measurement and management device 1700 includes an analyte measurement module 1702 configured to measure an analyte (e.g., blood glucose) in a bodily fluid sample (such as blood), a memory module 1704, processor module 1706, a visual display 1708, and a delivery device communication module 1710, in addition to a user interface 1712. The analyte memory module 1702, memory module 1704, processor module 1706, visual display 1708, delivery device communication module 1710 and user interface 1712 are in operative communication with one another.

Memory module 1704 is configured for storing at least one therapeutic administration protocol while processor module 1706 is configured to calculate a recommended therapeutic agent dosage and recommended administration time for user-activated delivery of the recommended therapeutic agent dosage. Such calculations use the therapeutic administration protocol stored in memory module 1704.

In addition, visual display module 1708 is configured to display the recommended therapeutic agent dosage and recommended administration time to a user and user interface 1712 is configured for accepting user input to analyte measurement and management device 1700 via, for example, user-operated interface buttons (not shown in FIG. 17).

Delivery device communication module 1710 is configured to detect user-activated administration (i.e., delivery) of the therapeutic agent by the user-activated therapeutic agent delivery device 1799 and communicate such detection to the processor module 1706 and/or memory module 1702. Moreover, the analyte measurement module, memory module, processor module, visual display, user interface and delivery device communication module of analyte measurement and management device 1700 are integrated as a single hand-held unit such as, without limitation, the unit illustrated in FIG. 1 as element 100.

Once apprised of the present disclosure, one of skill in the art will recognize that analyte measurement and management device 1700 can be modified to perform any of the functions described above with respect to FIGS. 1 through 16 and the devices and methods associated with these Figures. Moreover, analyte measurement and management device 1700 can be configured to possess characteristics described elsewhere herein with respect to embodiments described and illustrated herein including, for example, characteristics of the methods for operating an analyte measurement device described with respect to FIGS. 2 through 16.

FIG. 18 is a flow diagram illustrating stages in a method 1800 for measuring and managing an analyte in a bodily fluid according to an embodiment described and illustrated herein. Method 1800 includes, at step 1810, storing at least one therapeutic administration protocol in a memory module of an analyte measurement and management device.

Method 1800 also includes measuring the analyte in the bodily fluid sample using an analyte measurement module of the device (see step 1820 of FIG. 18) and calculating, with a processor module of the device, a recommended therapeutic agent dosage (for example, an insulin dosage) and a recommended administration time for user-activated delivery of the dosage. The calculation employs the therapeutic administration protocol loaded into the memory from an external source module (as noted in steps 1810-1830 of FIG. 18).

Method 1800 further includes displaying the recommended therapeutic agent dosage and administration time to a user on a visual display of the device as noted in step 1840, delivering a therapeutic agent dosage to the user via a user-activated therapeutic agent delivery device (see step 1850), and detecting the user-activated administration (delivery) of the therapeutic agent using a delivery device communication module of the device (refer to step 1860 of FIG. 18).

Moreover, at step 1870, method 1800 further includes communicating the aforementioned detection to the processor module and/or memory module using the delivery device communication module. It should be noted that the method employs analyte measurement, memory, processor, and delivery device modules, as well as a visual display, and user interface, that are integrated as a single hand-held unit (such as the unit depicted as element 100 in FIG. 1 and analyte measurement and management device 1700 of FIG. 17).

Once apprised of the present disclosure, one of skill in the art will recognize that method 1800 can be augmented to include performance of any of the functions described above with respect to FIGS. 1 through 17 and/or to have perform steps with characteristics described elsewhere herein with respect to various embodiments described and illustrated herein.

Embodiments of the current invention are beneficial in significantly reducing obstacles associated with initiating, maintaining and managing an analyte testing and therapeutic agent dosing regimen such as blood glucose monitoring and insulin administration. The present invention enables easy initiation and intensification, and improved compliance with a prescribed regimen by providing a simple, efficient way of guiding the patient in a step-by-step manner. By logging information on recommended versus the actual regimen followed by the patient in the manner described herein, the testing device and methods described and illustrated herein provide an effective and unitary record keeping system to help the patient and healthcare practitioner provide better care.

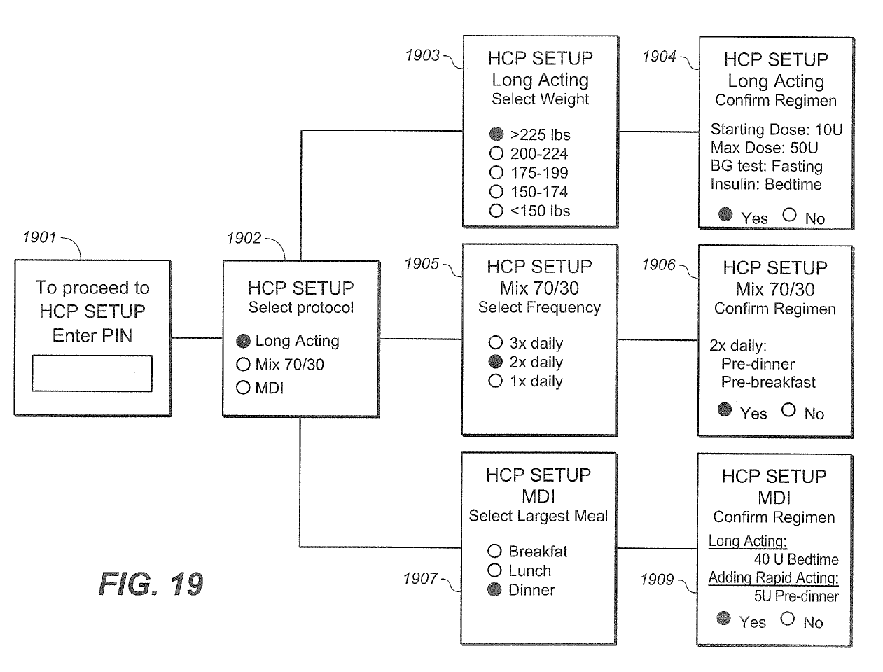

By virtue of the embodiments described and illustrated herein, a method of managing diabetes can be utilized with clinical benefit for persons with diabetes. In one example, as shown in the various display screens of FIGS. 19-22, a health care provider (“HCP”) can prepare to set up a therapeutic protocol in the measurement device 100 by logging in to an HCP selection menu (FIG. 19) by entry of a password (as shown at screen 1901), or for greater security, via the use of a cryptographic security key such as, for example, a USB security PKI token 11. Alternatively, the logging in process can be conducted via a secure remote terminal or computer 13 and performing the menu selection remotely via a HCP computer. Upon successful log in, the HCP can select one of a plurality of therapeutic protocols in screen 1902, such as, for example “Long-Acting” protocol; “Mix” protocol or Multiple Daily Injection (“MDI”) protocol.

Where the protocol selected is the Long-Acting protocol, the HCP would select the weight range of the user at screen 1903 and confirm at screen 1904 that the starting and maximum doses are correct with the preferred blood glucose test being performed after fasting and the insulin being delivered to the user's body at bedtime. Thereafter, the protocol is then transferred, by cables or via short or long-range wireless connection to the user's device 100.

Where the protocol selected is the Mix protocol in screen 1902, the HCP would select the frequency of insulin delivery over a fixed time period at screen 1905. At screen 1906, the HCP would need to confirm the insulin regimen as being of the selected frequency over a fixed duration but at specified time in a day. Thereafter, the protocol is then transferred, by cables or via short or long-range wireless connection to the user's device 100.

Where the protocol selected is the MDI protocol in screen 1902, the HCP would select the largest meal that the user would have during the day at screen 1907 and confirm at screen 1909 the regimen with the required dosages for rapid acting at specified daily event and rapid acting at a different daily event. Thereafter, the protocol is then transferred, by cables or via short or long-range wireless connection to the user's device 100.

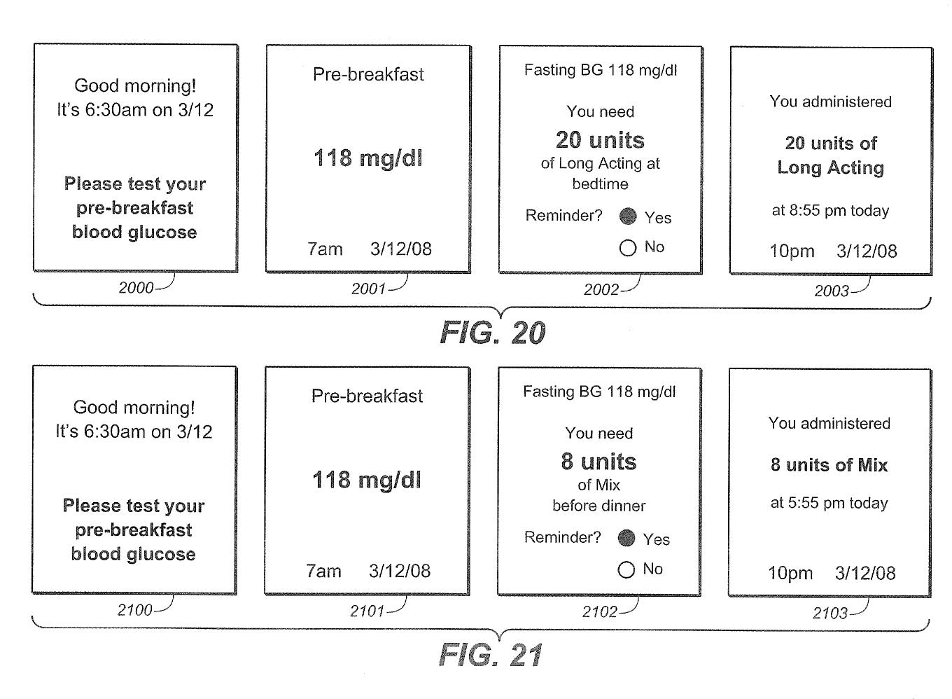

At device 100, the user whose HCP has selected a Long-Acting protocol would see a series of interactive screens in FIG. 20. At screen 2000, the processor 1706 would generate a greeting message and a reminder consistent with the protocol, which has been transferred from HCP computer 13 to the memory 1704. At this point the user should perform a blood glucose test using test strip 10. Upon analysis, the device would provide an output of the measured glucose concentration on screen 2001. Thereafter, the processor would generate a message at screen 2002 indicating the dosage needed for the physiological requirements of the user. At screen 2002, the user is given the option of selecting a reminder of when to take the required dosage of therapeutic agent. At screen 2002, it is preferred that the default selection is that of a reminder being activated. At the option of the user, various screens can be generated to provide a summary of blood glucose test, trends, therapeutic type and dosage taken. In one example, as shown in screen 2003, a summary of the therapeutic agent and the type of therapeutic agent taken at a particular time and date is provided.

At device 100, the user whose HCP has selected a Mix protocol would see a series of interactive screens in FIG. 21. At screen 2100, the processor 1706 would generate a greeting message and a reminder consistent with the protocol, which has been transferred from HCP computer 13 to the memory 1704. At this point the user should perform a blood glucose test using test strip 10. Upon analysis, the device would provide an output of the measured glucose concentration on screen 2101. Thereafter, the processor would generate a message at screen 2102 indicating the dosage needed for the physiological requirements of the user. At screen 2102, the user is given the option of selecting a reminder of when to take the required dosage of therapeutic agent. At screen 2102, it is preferred that the default selection is that of a reminder being activated. At the option of the user, various screens can be generated to provide a summary of blood glucose test, trends, therapeutic type and dosage taken. In one example, as shown in screen 2103, a summary of the therapeutic agent and the type of therapeutic agent taken at a particular time and date is provided.

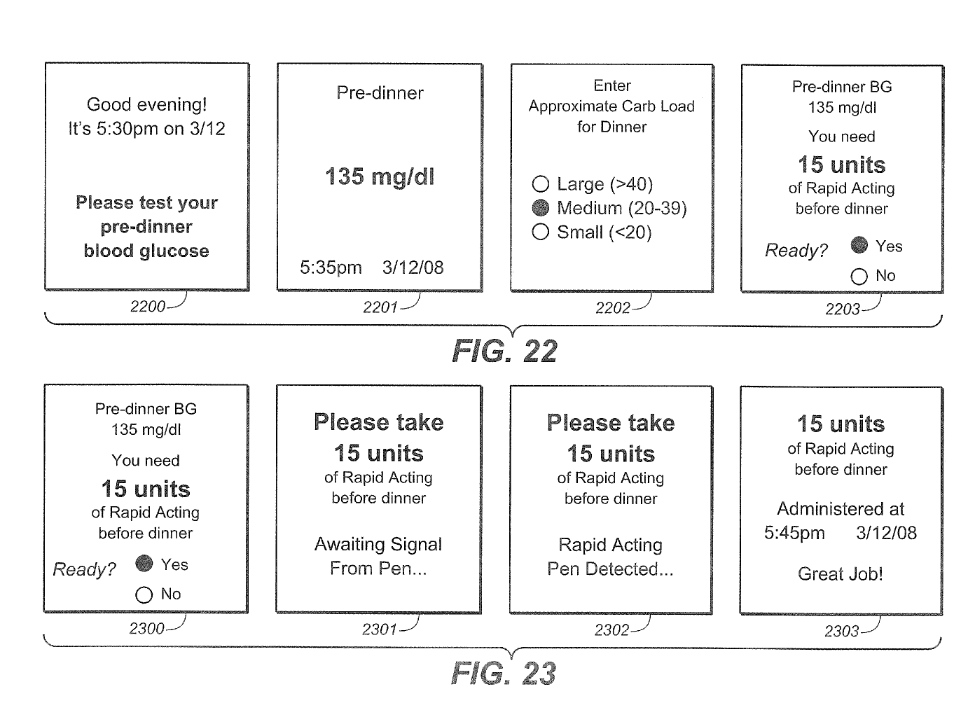

At device 100, the user whose HCP has selected a MDI protocol would see a series of interactive screens in FIG. 22. At screen 2200, the processor 1706 would generate a greeting message and a reminder consistent with the protocol, which has been transferred from HCP computer 13 to the memory 1704. At this point the user should perform a blood glucose test using test strip 10. Upon analysis, the device would provide an output of the measured glucose concentration on screen 2201. Thereafter, the processor would generate a message at screen 2202 indicating the dosage needed for the physiological requirements of the user. At screen 2202, the user is given the option of selecting a reminder of when to take the required dosage of therapeutic agent. At screen 2202, it is preferred that the default selection is that of a reminder being activated. At the option of the user, various screens can be generated to provide a summary of blood glucose test, trends, therapeutic type and dosage taken. In one example, as shown in screen 2203, a summary of the therapeutic agent and the type of therapeutic agent taken at a particular time and date is provided.

To ensure that the user follow the therapeutic regimen, the device 100 in conjunction with the therapeutic agent delivery 12 can be used to ensure compliance of the regimen by, as shown in FIG. 23, reminding the user of the therapeutic agent dosage needed based on the measured pre-meal blood glucose value at screen 2300, prompting the user at the specified time to deliver the required dosage for the user at screen 2301. The device 100 can be configured to detect activation of the therapeutic agent delivery device 12 or delivery of the therapeutic agent. Upon detection of activation of the device 12 (to infer delivery of therapeutic agent) or actual delivery of the therapeutic agent by transmission of a wireless signal from the delivery device 12 at screen 2302 to the measurement device 100, a message can be provided at screen 2302 to indicate the dosage and time of the administration of the therapeutic agent.

Applicants note that while the measurement device and delivery device have been described preferably as separate components, both components described and illustrated herein can be integrated into a unitary device with for example a delivery mechanism at one end of a unitary housing and a measuring device at the other end of the unitary housing. Alternatively, one component (e.g., delivery device or measurement device described and illustrated herein) could be mated or enclosed in the other component (e.g., delivery device or measurement device described and illustrated herein) with direct communication (e.g., wired or Infrared) between the components when both are mated together and via wireless communication when both components are separated.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A diabetes management system comprising:
an analyte measurement device including:
a housing;
a processor and memory disposed in the housing;
a measurement unit in communication with the processor to provide a numerical value representing generally an amount of analyte in body fluids;
a display in communication with the processor to display information relating to analyte and the therapeutic agent;
a first wireless module coupled to the processor and memory to store data received by the first wireless module in the memory;
a therapeutic agent delivery device including:
a delivery device housing;
a delivery mechanism disposed in the housing that delivers a dosage of the agent to the user upon actuation by the user or health care provider;
a second wireless module disposed in the housing, the second wireless module being configured to automatically, without prompting or any action from a user, transmits a signal to the first wireless module indicative of:
(a) type of therapeutic agent delivered; and
(b) amount of therapeutic agent delivered to the user; or
(c) type of therapeutic agent device from which the therapeutic agent was administered.

2. A diabetes management system comprising:
an analyte measurement device including:
a housing;
a processor and memory disposed in the housing;
a measurement unit in communication with the processor to provide a numerical value representing generally an amount of analyte in body fluids;
a display in communication with the processor to display information relating to analyte and the therapeutic agent;
a first wireless module coupled to the processor and memory to store data received by the first wireless module in the memory;
a therapeutic agent delivery device including:
a delivery device housing;
a delivery mechanism disposed in the housing that delivers a dosage of the agent to the user upon actuation by the user or health care provider;
a second wireless module disposed in the housing and coupled to the delivery mechanism, the second wireless module, upon prompting or by an action from a user, transmits a signal to the first wireless module indicative of:
(a) type of therapeutic agent delivered; and
(b) amount of therapeutic agent delivered to the user; or
(c) type of therapeutic agent device from which the therapeutic agent was administered; and
a health care provider computer in communication with the analyte measurement device to prescribe one of a plurality of therapeutic agent titration protocols to the memory of the analyte measurement device.

3. A diabetes management device comprising:
a housing;
a processor and memory disposed in the housing, the memory including a plurality of therapeutic administration protocols loaded into the memory from an external source that relates a dosage administration to one or more analyte amount;
a measurement unit in communication with the processor to provide a numerical value representing generally an amount of analyte in body fluids;
a display in communication with the processor to display information relating to measured amount of analyte and type of therapeutic agent from the plurality of protocols.

4. The diabetes management device of claim 3, further comprising a first wireless module coupled to the processor and memory to store data received by the first wireless module in the memory; and

5. The diabetes management device of claim 4, further comprising a therapeutic agent delivery device including:
a delivery device housing;
a second wireless module disposed in the housing and configured the second wireless module upon prompting or by an action from a user, transmits a signal to the first wireless module indicative of:
(a) type of therapeutic agent delivered; and
(b) amount of therapeutic agent delivered to the user; or
(c) type of therapeutic agent device from which the therapeutic agent was administered.

6. The diabetes management system of one of claims 1 and 2, in which the processor is configured to utilize the numerical value so as to provide dosage recommendation for delivery of therapeutic agent according to one of a plurality of recommended administration protocols.

7. The diabetes management system of one of claims 1 and 2, in which the type of therapeutic agent comprises long-acting insulin, a mixture of different types of insulin, rapid-acting insulin or combinations thereof.

8. The diabetes management system of claim 1 or claim 2, in which the display provides an indication of the user's analyte measurement values and therapeutic agent administration history.

9. The diabetes management device of claim 3, further comprising a health care provider computer in communication with the device to prescribe one of a plurality of therapeutic agent titration protocols to the memory of the device.

10. The diabetes management system of claim 8, in which the plurality of therapeutic agent titration protocols comprise basal initiation, titration, or non-carbohydrate counting multiple-daily-injections for type 2 diabetes users.

11. The diabetes management system of claim 8, in which the health care provider's computer receives inputs relating to the user of the analyte measurement device including:
(a) body weight;
(b) frequency of insulin
(c) dosing of insulin.

13. The diabetes management system of claim 10, further comprising an audio output in communication with the processor.

14. The diabetes management system of claim 10, in which the processor is programmed to generate:
(a) a reminder for the user to perform an analyte measurement;
(b) a reminder for the user to perform therapeutic agent delivery to the user in accordance with the recommended administration protocol;
(c) instructions on an amount of therapeutic agent to be used by the user based generally on at least one analyte value as measured by the analyte measurement device; and
(d) an indicator of the user's adherence to the recommended administration protocol.

15. The diabetes management system of claim 1 or claim 2, in which the analyte comprises blood glucose and the bodily fluid comprises blood.

16. The diabetes management system of claim 1 or claim 2, in which the analyte measurement device includes a strip port configured to operatively accommodate an analyte test strip.

17. The diabetes management system of claim 1 or claim 2, in which the therapeutic administration protocol comprises at least one of a recommended dosage of the therapeutic agent, type of therapeutic agent, and administration time of the therapeutic agent.

18. The diabetes management system of claim 1 or claim 2, in which the therapeutic administration protocol employs a result of at least one analyte measurement stored in the memory to provide the recommended therapeutic agent dosage and recommended administration time.

19. The diabetes management system of claim 1 or claim 2, in which the memory comprises storage of analyte measurements made by the analyte measurement device along with associated dates and times, the recommended therapeutic agent dosage, the recommended administration time and information related to the actual therapeutic agent dosage and actual administration time in the memory.

20. The diabetes management system of claim 1 or claim 2, in which the memory comprises storage of at least one flag that indicates when analyte measurements and therapeutic agent dosages occurred in relationship to user meals in the memory.

21. The diabetes management system of claim 1 or claim 2, further including an alarm module configured to alert a user when the medical delivery device communication module has not detected user-activated administration of the therapeutic agent.

22. The diabetes management system of claim 1 or claim 2, in which the therapeutic agent is selected from the group consisting essentially of medications for diabetes, medications for metabolic management, inflammation management, hormonal therapy agents, oncology agents, pain management agents, regenerative medicine agents, and a combination thereof.

23. The diabetes management system of claim 1 or claim 2, further comprising a user interface on one of the analyte measurement device and the analyte delivery device, and in which the processor and display are configured to prompt a user to confirm administration of therapeutic agent via the user interface.

24. The diabetes management system of claim 1 or claim 2, in which the memory comprises storage to store date and time in a user-selected time zone; a compliance table that includes at least a record of missed therapeutic administrations; and an actual initiation date of the therapeutic protocol.

25. The diabetes management system of claim 1 or claim 2, in which each of the first and second wireless module comprises a transmitter and receiver module.

26. The diabetes management system of claim 1 or claim 2, in which the first wireless module comprises a transmitter and receiver module and the second wireless module comprises a transmitter.

27. The diabetes management system of claim 1 or claim 2, in which the therapeutic administration protocol comprises treat to target, 4T Titration Protocol, insulation titration or Basal/Bolus Titration protocol.

28. A method of managing diabetes of a user, the method comprising:
selecting a therapeutic administration protocol in accordance with therapeutic requirements of the diabetic user;
transferring the therapeutic administration protocol to an analyte measurement device assigned to the user;
confirming delivery of therapeutic agent to the user in accordance with the therapeutic administration protocol; and
generating a plurality of prompts to the user including:
(a) a reminder to measure analyte at a specified time;
(b) a reminder to administer a recommended dosage of therapeutic agent within a specified time frame; and
(c) a report of compliance of the user to the therapeutic administration protocol.

29. The method of claim 28, in which the selecting comprises:
entering a set up mode;
selecting one of a plurality of therapeutic administration protocols, and upon selection of:
(a) a long acting protocol, selecting a body weight range and confirming a starting dosage, maximum dosage, fasting measurement and specified time for delivery of the therapeutic agent;
(b) a premix protocol, selecting a frequency of delivery of therapeutic agent and confirming the frequency and specified time for delivery of the therapeutic agent; or
(c) a multiple daily administration protocol, selecting a largest meal during a specified time duration for rapid acting therapeutic agent initiation and confirming the dosage of a long acting therapeutic agent at a specified time.

30. The method of claim 28, in which the generating comprises displaying at least one of:
(a) a result of the analyte measurement;
(b) the dosage specified; or
(c) a summary of the dosage administered at one or more specified time slots;
(d) a summary of analyte measurements; and
(e) a summary of missed dosing and missed analyte measurements.

31. The method of claim 28, in which the confirming comprises prompting the user to confirm delivery of the agent.

32. The method of claim 28, in which the confirming comprises automatically storing, without prompting the user, the dosage of agent delivered and approximate time of delivery.

33. A method of operating an analyte measurement device having a plurality of therapeutic administration protocols stored in a memory of the device, the memory in communication with a processor, the processor configured to interface with user inputs and provide various output information, the method comprising:
accessing a selection menu generated by the processor with protocols loaded into the memory from an external source;
selecting one therapeutic administration protocol from the plurality of therapeutic administration protocols; and
outputting dosage information for therapeutic agent to be administered to a user based on one or multiple analyte amounts or concentration values stored in the memory.

34. The method of claim 33, in which the accessing comprises:
logging in to the selection menu via a password protected interface.

35. The method of claim 33, in which the accessing comprises:
logging in to the selection menu via a security token.
